(19) United States
(12) Patent Application Publication
(10) Pub. No.: US 2005/0065499 A1
(43) Pub. Date: Mar. 24, 2005

(54) Low-profile catheter valve

(76) Inventors: Nareak Douk, Lowell, MA (US); Nasser Rafiee, Andover, MA (US)

Correspondence Address: Catherine C. Maresh, Medtronic Vascular, Inc., 3576 Unocal Place, Santa Rosa, CA, 95403, US

(21) Appl. No.: 10/665,647

(22) Filed: Sep. 19, 2003

(51) Int. Cl. A61M 25/16

(52) U.S. Cl. 604/537; 604/247

(57) ABSTRACT

A catheter having a low-profile valve. The catheter has a central lumen and includes a plurality of longitudinal struts and longitudinal apertures interspaced around the circumference of a proximal portion of the catheter. A self-sealing polymer is disposed on at least a portion of each strut, separably sealing the struts one to another. The struts separate to allow passage of a fluid into or out of the central lumen of the catheter and reseal to prevent passage of a fluid into or out of the central lumen.

### TECHNICAL FIELD

This invention relates generally to biomedical devices that are used for treating vascular conditions. More specifically, the invention relates to a low-profile catheter valve.

### BACKGROUND OF THE INVENTION

Guidewires are conventionally used to guide medical instruments to a desired treatment location within a patient's vasculature. In a typical procedure, the clinician forms an access point for the guidewire by creating an opening in a peripheral blood vessel, such as the femoral artery. The highly flexible guidewire is then introduced through the opening and is advanced by the clinician through the patient's blood vessels until the guidewire extends across a vessel segment to be treated. A treatment catheter, such as a balloon catheter for a percutaneous transluminal coronary angioplasty (PTCA), may then be inserted over the guidewire and similarly advanced through vasculature until it reaches the treatment site.

In certain treatment procedures, it is desirable to serially advance and withdraw a number of different treatment catheters over a single guidewire that has been placed in a particular location. Typically, a first treatment catheter is advanced over the guidewire, withdrawn, and then fully removed from the portion of the guidewire that extends out of the patient's vessel. The guidewire is then available to act as a guide for a different treatment catheter.

It is sometimes advantageous to equip the distal end of a guidewire with at least one inflatable balloon, either to provide temporary occlusion of a vessel or to anchor the guidewire within a vessel. Anchoring the guidewire helps to prevent the guidewire from being displaced from its position while treatment catheters are advanced or withdrawn over the placed guidewire. An occlusion guidewire can be used as “distal protection” to prevent debris generated during vessel treatment from moving with the flowing blood to embolize distally.

A permanent inflation manifold, of the type used with conventional catheters having an inflatable balloon, would prevent treatment catheters from being exchanged one for another over an occlusion guidewire. Therefore, a removable inflation manifold and a valve to maintain the balloon in the inflated state are desirable for an occlusion guidewire. U.S. Pat. No. 5,167,239 to Cohen et al. discloses one such device. However, the valve apparatus used by the Cohen device is relatively bulky, having an outer diameter in its preferred embodiment of 0.0355 inches. As can be readily appreciated, the diameter of the valve on a guidewire dictates the inner diameter and, consequently, the outer diameter of a treatment catheter introduced over the valve. Therefore, it would be desirable to provide a low-profile catheter valve that overcomes the aforementioned and other disadvantages.

### SUMMARY OF THE INVENTION

One aspect of the present invention is a low-profile catheter valve, comprising a catheter and a self-sealing polymer. The catheter has a central lumen and includes a plurality of longitudinal struts and longitudinal apertures interspaced around the circumference of a proximal portion of the catheter. The self-sealing polymer is disposed on at least a portion of each strut, separably sealing the struts one to another. The struts separate to allow passage of a fluid into or out of the central lumen of the catheter and reseal to prevent passage of a fluid into or out of the central lumen.

Another aspect of the present invention is a system for treating a vascular condition, comprising a catheter, a self-sealing polymer, and an inflatable balloon. The catheter has a central lumen and includes a plurality of longitudinal struts and longitudinal apertures interspaced around the circumference of a proximal portion of the catheter. The self-sealing polymer is disposed on at least a portion of each strut, separably sealing the struts one to another. The inflatable balloon is operably attached to a distal portion of the catheter. The struts separate to allow inflation of the balloon through the central lumen of the catheter, reseal to maintain inflation, and separate to allow deflation of the balloon.

Yet another aspect of the present invention is a method for manufacturing a low-profile catheter valve. A plurality of longitudinal apertures and longitudinal struts are formed into a proximal portion of a catheter. A self-sealing polymer is applied to at least a portion of each strut.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of a low-profile catheter valve, in accordance with the present invention;

FIG. 2 is a transverse cross-sectional view of the low-profile catheter valve of FIG. 1;

FIG. 3 is a longitudinal cross-sectional view of an adaptor used to manipulate the low-profile catheter valve of FIG. 1 and FIG. 2;

FIG. 4 is an illustration of an alternative embodiment of a low-profile catheter valve, in accordance with the present invention;

FIG. 5 is a transverse cross-sectional view of the low-profile catheter valve of FIG. 4;

FIG. 6 is a longitudinal cross-sectional view of an alternative adaptor used to manipulate the low-profile catheter valve of FIG. 4 and FIG. 5;

FIG. 7 is an illustration of one embodiment of a system for treating a vascular condition, in accordance with the present invention;

FIG. 8 is an illustration of another embodiment of a system for treating a vascular condition, in accordance with the present invention; and

FIG. 9 is a flow diagram of one embodiment of a method for making a low-profile catheter valve, in accordance with the present invention.

### DETAILED DESCRIPTION OF THE PRESENTLY PREFERRED EMBODIMENTS

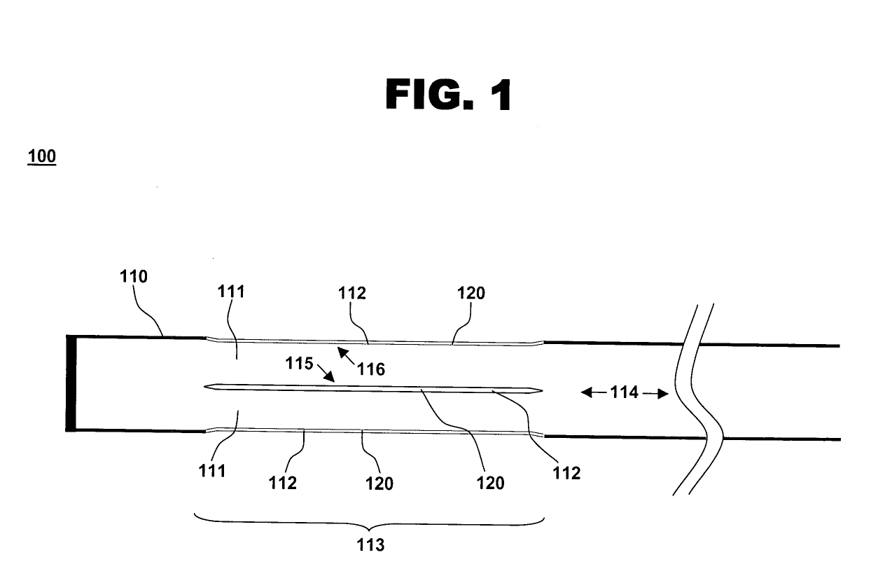

One aspect of the present invention is a low-profile catheter valve. One embodiment of the valve, in accordance with the present invention, is illustrated in FIG. 1 at 100. Valve 100 comprises catheter 110 and self-sealing polymer 120. Catheter 110 includes a plurality of longitudinal struts 111 and longitudinal apertures 112 interspaced around the circumference of proximal portion 113. Catheter 110 also includes central lumen 114. Each strut has two side surfaces, first side surface 115 and second side surface 116.

Catheter 110 may be, for example, a hollow guidewire and may include an inflatable balloon (not shown) operably attached to a distal portion of the catheter. Where catheter 110 is to be used as a guidewire during a procedure such as a conventional percutaneous transluminal coronary angioplasty (PTCA) involving femoral artery access, catheter 110 may be about centimeters to about 300 centimeters long, with a length of about 180 centimeters often being used. The outer diameter of the catheter may range from about 0.010 inches to 0.038 inches, and preferably is 0.014 inches or smaller when the catheter is to be used as a guidewire. Catheter 110 may be made of an appropriate biocompatible material such as nitinol.

Longitudinal apertures 112 may be, for example, 5 to 7 millimeters long and, in this embodiment, are preferably narrowly eye-shaped, an intersection of two circles forming two arcs with a common chord. Longitudinal struts 111 result when apertures 112 are formed into catheter 110.

Self-sealing polymer 120 is disposed on at least one side surface of each strut. Polymer 120 separably seals first side surface 115 of each strut to second side surface 116 of the adjoining strut, thereby separably sealing struts 111 one to another and closing apertures 112. Self-sealing polymer 120 may additionally be disposed on one or both of the inner and outer surfaces of the struts.

Self-sealing polymer 120 may be polyurethane, silicone, a suitable biocompatible polymer, or the like. Where catheter 110 is to be used as a guidewire, it may be desirable for self-sealing polymer 120 to be hydrophilic to ensure that, when valve 100 has been wetted, a second catheter may pass easily there over.

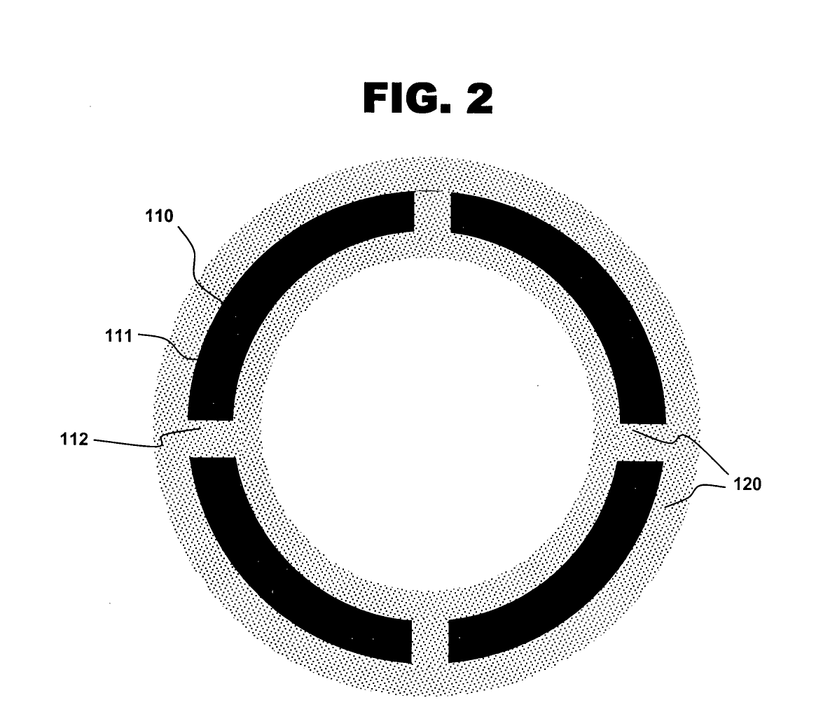

FIG. 2, in which like elements share like numbers with FIG. 1, is a transverse cross-sectional view of proximal portion 113 of valve 100, showing longitudinal struts 111 interspaced with longitudinal apertures 112. Self-sealing polymer 120 is shown in FIG. 2 disposed on not only the sides of the struts, but also on the inner and outer surfaces of the struts.

While the present embodiment is illustrated in FIG. 1 and FIG. 2 as including four struts interspaced with four narrowly eye-shaped apertures, it will be apparent to one skilled in the art that factors such as the number, shape, size, and spacing of the longitudinal struts and longitudinal apertures may be varied as desired.

Struts 111 separate to allow passage of a fluid into or out of central lumen 114 through apertures 112 and reseal to prevent passage of the fluid into or out of central lumen 114. In this embodiment, the proximal end of catheter 110 is sealed.

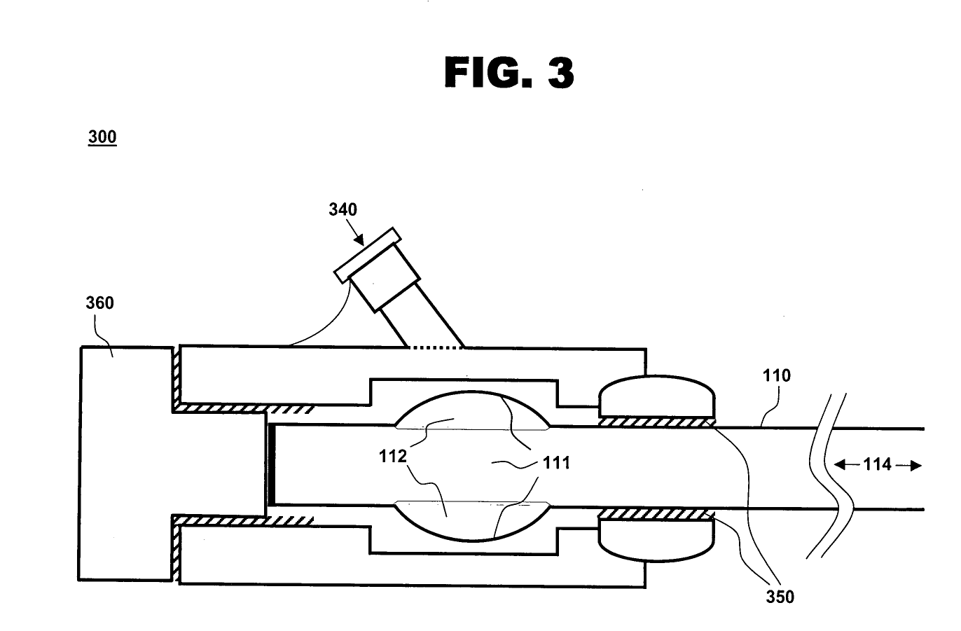

Struts 111 may separate in response to applying a mechanical force to proximal portion 113 and reseal in response to withdrawing the mechanical force. An adaptor such as that shown in FIG. 3, in which like elements share like numbers with FIG. 1 and FIG. 2, may be used to apply and withdraw the mechanical force. Adaptor 300 may be removably mounted about proximal portion 113 as seen in FIG. 3. Adaptor 300 is movable between a first position in which a mechanical force is applied to proximal portion 113 and a second position in which the mechanical force is withdrawn. The adaptor is in fluid communication with a fluid delivery device through opening 340 and has at least one seal 350 that engages the circumference of catheter 110 distal to proximal portion 113. When engaged, seal 350 establishes a fluid-tight chamber surrounding proximal portion 113. Additional seals and gaskets may be used to ensure that the adaptor itself is fluid tight, particularly around proximal knob 360.

FIG. 3 shows adaptor 300 in the first position, with a mechanical force applied to proximal portion 113 using proximal knob 360. Applying the mechanical force to proximal portion 113 axially compresses proximal portion 113 and bows struts 111 outward, separating the struts and opening apertures 112. A fluid may now flow into adaptor 300 through opening 340 of the adaptor and then into central lumen 114 through apertures 112. When the mechanical force is withdrawn, for example by unscrewing or pulling out proximal knob 360, proximal portion 113 returns to an axially uncompressed condition and struts 111 reseal, preventing passage of the fluid out through the valve.

Where catheter 110 is to be used as a guidewire having an inflatable balloon at its distal end, separating struts 111 to admit a fluid permits inflation of the balloon. Resealing the struts maintains inflation of the balloon. Once struts 111 have resealed, adaptor 300 may be removed and various treatment catheters may be serially advanced and withdrawn over the guidewire. To deflate the balloon, adaptor 300 may be remounted on proximal portion 113, and a mechanical force may be reapplied to separate struts 111 and permit the inflation fluid to pass out through apertures 112 and opening 340.

One skilled in the art will recognize that a wide variety of adaptors may be appropriate for the present embodiment of the invention. For example, an alternative adaptor may have seals that engage the circumference of the catheter both proximal and distal to the longitudinal struts and apertures. In this example, the sealed proximal end of the catheter extends outside of the adaptor, and engaging the first and second seals establishes a fluid-tight chamber surrounding only the portion of the catheter that includes struts 111 and apertures 112. The adaptor is movable between a first position in which a mechanical force is applied to proximal portion 113 and a second position in which the mechanical force is withdrawn. In the first position, the adaptor shortens longitudinally, axially compressing proximal portion 113, bowing out struts 111, and opening apertures 112. In the second position, proximal portion 113 returns to an axially uncompressed condition, allowing struts 111 to reseal and thereby preventing passage of the fluid out through the valve.

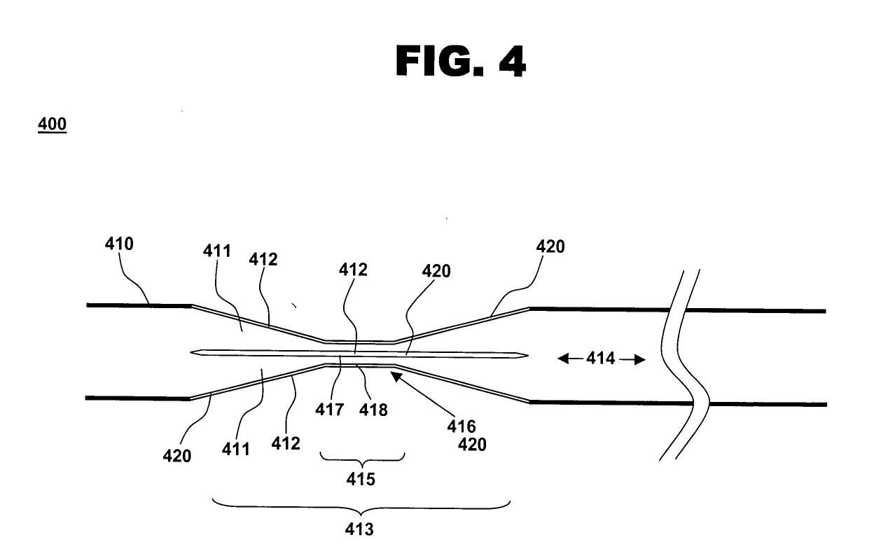

Another embodiment of the low-profile catheter valve, in accordance with the present invention, is illustrated in FIG. 4 at 400. Valve 400 comprises catheter 410 and self-sealing polymer 420. Catheter 410 includes a plurality of longitudinal struts 411 and longitudinal apertures 412 interspaced around the circumference of proximal portion 413. Struts 411 are radially deformed into central lumen 414 of catheter 410 such that necked-down or narrowed region 415 is formed, this narrowed region including narrowed lumen 416. Each strut has two side surfaces, first side surface 417 and second side surface 418.

Catheter 410 may be, for example, a hollow guidewire and may include an inflatable balloon (not shown) operably attached to a distal portion of the catheter. Where catheter 410 is to be used as a guidewire during a procedure such as a conventional percutaneous transluminal coronary angioplasty (PTCA) involving femoral artery access, catheter 410 may be about 150 centimeters to about 300 centimeters long, with a length of about 180 centimeters often being used. The outer diameter of the catheter may range from about 0.010 inches to 0.038 inches, and preferably is 0.014 inches or smaller when the catheter is to be used as a guidewire. Catheter 410 may be made of an appropriate biocompatible material such as nitinol.

Catheter 410 includes longitudinal struts 411 and longitudinal apertures 412, which are interspaced around the circumference of proximal portion 413. Apertures 412 may be, for example, 5 to 7 millimeters long. When struts 411 are radially deformed into central lumen 414 of catheter 410, apertures 412 are narrowed, and narrowed region 415 is formed. Elongate hexagonal apertures are preferred for this embodiment because, when struts 411 are radially deformed into central lumen 414, hexagonal apertures produce a narrowed region having a uniformly narrowed lumen.

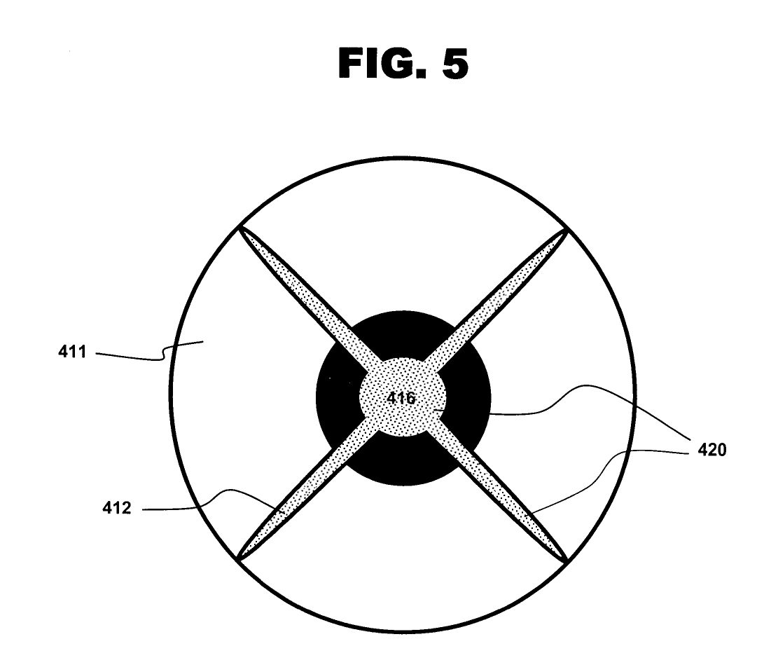

FIG. 5, in which like elements share like numbers with FIG. 4, is a transverse cross-sectional view of valve 400, showing narrowed lumen 416, which results when struts 411 are radially deformed into central lumen 414. Struts 411 may be heat set to maintain or “memorize” the shape of their radial deformation into central lumen 414.

Self-sealing polymer 420 is disposed on side surfaces 417 and 418 of each longitudinal strut. First side surface 417 of each strut is separably sealed to second side surface 418 of the adjoining strut, thereby closing apertures 412 and separably sealing struts 411 one to another. Self-sealing polymer 420 is additionally disposed on an inner surface of each strut, separably sealing the inner surface of the strut to the inner surface of at least one opposing strut, thereby closing narrowed lumen 416.

Struts 411 separate to allow passage of a fluid into or out of central lumen 414 and reseal to prevent passage of the fluid into or out of lumen 414. In this embodiment, the proximal end of catheter 410 is not sealed, and struts 411 may separate in response to inserting a hollow needle into narrowed lumen 416 through the proximal end of catheter 410. When the needle is inserted, struts 411 separate enough to allow passage of the needle but not enough to cause apertures 412 to gap open. The needle may be inserted completely through narrowed lumen 416 and into central lumen 414, thereby allowing passage of a fluid through the needle and into central lumen 414. Alternatively, the needle may be inserted only as far into narrowed lumen 416 as is necessary to cause struts 411 to separate and narrowed lumen 416 to open. In this second example, the fluid passes through the needle, through a portion of narrowed lumen 416, and into central lumen 414.

The polymer sealing the sides of struts 411 together may allow apertures 412 to widen, while still preventing the apertures from gapping when a needle is inserted into narrowed lumen 416. To provide further assurance that apertures 412 will not gap open when a needle is inserted, self-sealing polymer 420 may be further disposed over the outer surface of narrowed region 415. In this example, when struts 411 resume their narrowed configuration, self-sealing polymer 420 fills apertures 412 and narrowed lumen 416 and also forms a layer of polymer over the outer surface of narrowed region 415.

Self-sealing polymer 420 may be polyurethane, silicone, a suitable biocompatible polymer, or the like. Where catheter 410 is to be used as a guidewire, it may be desirable for self-sealing polymer 420 to be hydrophilic to ensure that, when valve 400 has been wetted, a second catheter may pass easily there over.

As another measure to prevent gapping or to increase ease of use, an elastic material (not shown) may be coated over the outer surface of narrowed region 415, either directly over the catheter material and any exposed self-sealing polymer or over a layer of self-sealing polymer disposed over the entire outer surface of narrowed region 415. The elastic material may be hydrophilic to ensure that, when valve 400 has been wetted, a second catheter may pass easily there over.

Where catheter 410 is to be used as a guidewire having an inflatable balloon at its distal end, separating struts 411 to admit a fluid permits inflation of the balloon. Struts 411 reseal in response to withdrawing the needle from narrowed lumen 416, maintaining inflation of the balloon. Once the needle has been withdrawn, various treatment catheters may be serially advanced and withdrawn over the guidewire. The needle may be reinserted to permit deflation of the balloon.

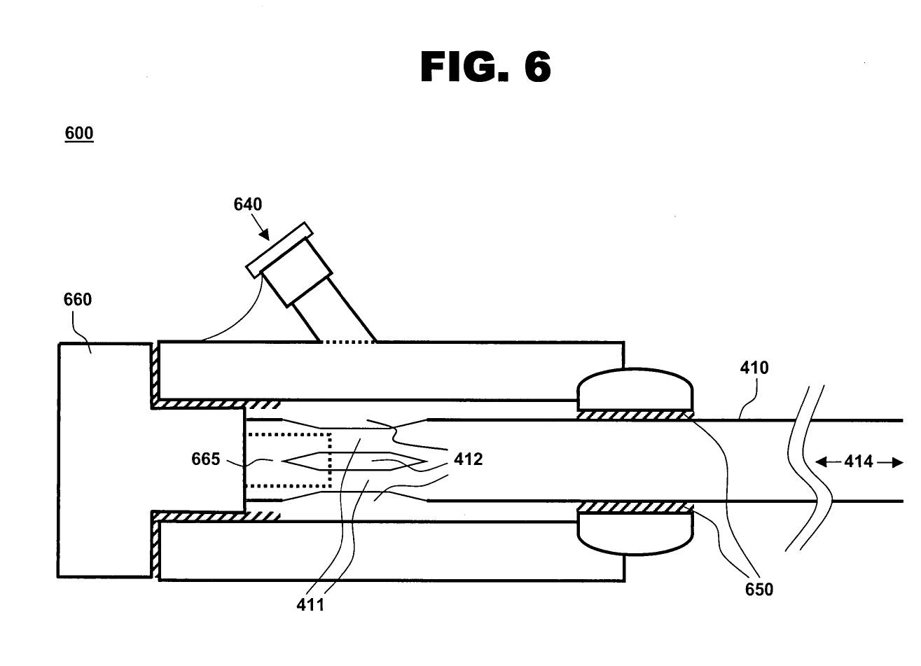

Struts 411 may also separate in response to applying a mechanical force to proximal portion 413 and reseal in response to withdrawing the mechanical force. An adaptor such as that shown in FIG. 6, in which like elements share like numbers with FIG. 4 and FIG. 5, may be used to apply and withdraw the mechanical force.

Adaptor 600 may be removably mounted about proximal portion 413 as seen in FIG. 6. Adaptor 600 is movable between a first position in which a mechanical force is applied to proximal portion 413 and a second position in which the mechanical force is withdrawn. The adaptor is in fluid communication with a fluid delivery device through opening 640 and has at least one seal 650 that engages the circumference of catheter 410 distal to proximal portion 413. When engaged, seal 650 establishes a fluid-tight chamber surrounding proximal portion 413. Additional seals and gaskets may be used to ensure that the adaptor itself is fluid tight. Rod 665 is attached to proximal knob 660 and protrudes into adaptor 600.

FIG. 6 shows adaptor 600 in the first position, with a mechanical force applied to proximal portion 413 using proximal knob 660. As seen in FIG. 6, when proximal knob 660 is pressed or screwed inward, rod 665 enters the proximal end of valve 400 and extends just far enough into narrowed lumen 416 to apply pressure to struts 411, thereby separating the struts and opening not only narrowed lumen 416, but also some or all of apertures 412. A fluid may now flow into adaptor 600 through opening 640 and through apertures 412 into central lumen 414. When rod 665 is withdrawn from narrowed lumen 416, for example by unscrewing or pulling out proximal knob 660, struts 411 reseal, closing apertures 412 and narrowed lumen 416, and preventing fluid from passing out of the valve. Once struts 411 have resealed, adaptor 600 may be removed and various treatment catheters may be serially advanced and withdrawn over the guidewire. Adaptor 600 may be remounted to allow the fluid to be withdrawn.

While the present embodiment as illustrated in FIGS. 4 through 6 includes four struts interspaced with four hexagonal apertures, it will be apparent to one skilled in the art that factors such as the number, shape, size, and spacing of the longitudinal struts and longitudinal apertures may be varied to control the shape and size of the narrowed region.

Valves 100 and 400 are discussed above in the context of an occlusion guidewire; however, one skilled in the art will recognize that these valves may be useful in connection with other catheters into which fluids are introduced or through which fluids are withdrawn. For example, the present invention would be useful in inflating and maintaining inflation of a flow-directed catheter, which includes a low-pressure, elastomeric balloon.

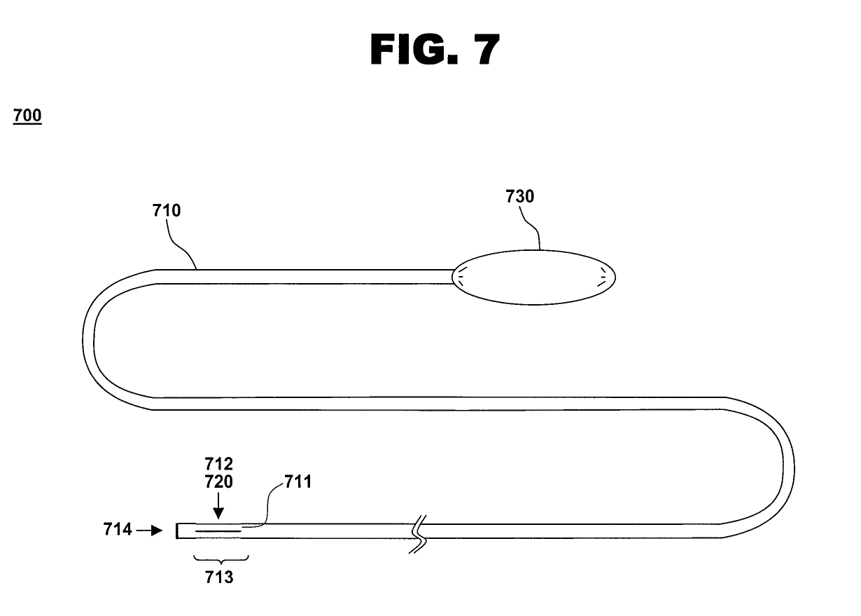

Another aspect of the present invention is a system for treating a vascular condition. One embodiment of the system, in accordance with the present invention, is illustrated in FIG. 7 at 700. System 700 comprises catheter 710, self-sealing polymer 720, and inflatable balloon 730. Catheter 710 includes a plurality of longitudinal struts 711 and longitudinal apertures 712 interspaced around the circumference of proximal portion 713. Catheter 710 also includes a central lumen 714. Self-sealing polymer 720 is disposed on at least a portion of each strut 711, separably sealing the struts one to another. Struts 711 separate to allow inflation of balloon 730 through central lumen 714 of catheter 710, reseal to maintain inflation, and separate again to allow deflation of the balloon.

Catheter 710 may be, for example, a hollow guidewire. Where catheter 710 is to be used as a guidewire for other catheters, for example in a conventional percutaneous transluminal coronary angioplasty (PTCA) procedure involving femoral artery access, catheter 710 may be about 150 centimeters to about 300 centimeters long, with a length of about 180 centimeters often being used. The outer diameter of the catheter may range from about 0.010 inches to 0.038 inches, and preferably is 0.014 inches in outer diameter or smaller when used as a guidewire. Catheter 710 may be made of an appropriate biocompatible material such as nitinol.

Self-sealing polymer 720 may be polyurethane, silicone, a suitable biocompatible polymer, or the like. Polymer 720 is disposed on at least the side surfaces of each strut 711. Polymer 720 separably seals a first side surface of each strut to a second side surface of the adjoining strut, thereby separably sealing the struts one to another and closing the intervening apertures 712, which may be narrowly eye-shaped. Self-sealing polymer 720 may additionally be disposed on one or both of the inner and outer surfaces of the struts.

Self-sealing polymer 720 may be polyurethane, silicone, a suitable biocompatible polymer, or the like. Where catheter 710 is to be used as a guidewire, it may be desirable for polymer 720 to be hydrophilic to ensure that, when polymer 720 has been wetted, a second catheter may pass easily there over.

In the present embodiment as seen in FIG. 7, the proximal end of catheter 710 is sealed. The system as depicted may be operated using an adaptor such as that shown in FIG. 3. The adaptor may be removably mounted on proximal portion 713 of catheter 710. The adaptor has a seal to engage the circumference of catheter 710 distal to proximal portion 713. Engaging the seal establishes a fluid-tight chamber surrounding proximal portion 713. The adaptor is movable between a first position in which a mechanical force is applied to proximal portion 713 and a second position in which the mechanical force is withdrawn. Applying the mechanical force to proximal portion 713 bows struts 711 outward, separating the struts and opening apertures 712. The adaptor is in fluid communication with a fluid delivery device, and a fluid can now flow into the adaptor and through apertures 712 and central lumen 714 to inflate balloon 730. When the mechanical force is withdrawn, struts 711 reseal to maintain inflation of the balloon. Once the struts have resealed, the adaptor may be removed and treatment catheters may be serially advanced and withdrawn over the guidewire. To deflate the balloon, the adaptor may be remounted and the force reapplied to separate struts 711 and allow the fluid to pass out through apertures 712.

Another adaptor for use with the present invention may have seals that engage the circumference of the catheter both proximal and distal to the longitudinal struts and apertures. Using this adaptor, the sealed proximal end of catheter 710 extends outside of the adaptor, and the first and second seals establish a fluid-tight chamber surrounding only the portion of the catheter that includes struts 711 and apertures 712. The adaptor is movable between a first position in which a mechanical force is applied to proximal portion 713 and a second position in which the mechanical force is withdrawn. In the first position, the adaptor shortens longitudinally, thereby applying a mechanical force to axially compress proximal portion 713, bowing struts 711 outward, and opening apertures 712. In the second position, proximal portion 713 returns to an axially uncompressed configuration, allowing struts 711 to reseal and apertures 712 to close, thus preventing flow of a inflation fluid into or out of system 700.

Inflatable balloon 730 is operably attached to a distal portion of catheter 710. Inflatable balloon 730 may be made of a suitable material such as thermoplastic polyurethane (TPU) resins, styrene-ethylene-butadiene-styrene (SEBS), PEBAX, or the like.

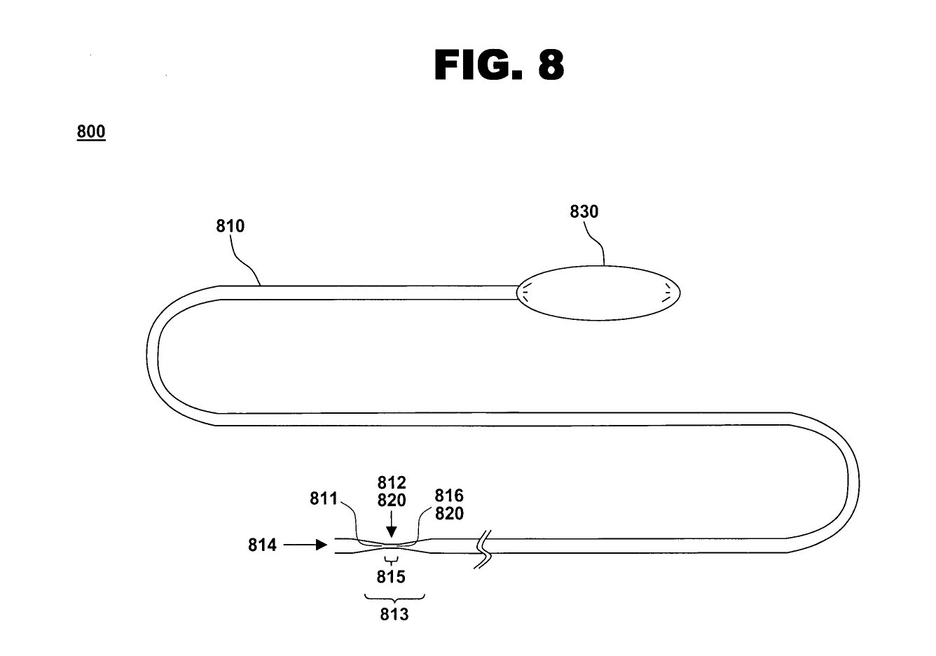

Another embodiment of a system for treating a vascular condition, in accordance with the present invention, is illustrated in FIG. 8 at 800. System 800 comprises catheter 810, self-sealing polymer 820, and inflatable balloon 830. Catheter 810 includes a plurality of longitudinal struts 811 and longitudinal apertures 812 interspaced around the circumference of proximal portion 813. Struts 811 are radially deformed into central lumen 814 of catheter 810 such that narrowed region 815 is formed in the catheter, narrowed region 415 including narrowed lumen 816.

Self-sealing polymer 820 may be polyurethane, silicone, a suitable biocompatible polymer, or the like. Polymer 820 is disposed on the side surfaces of each strut 811. Polymer 820 separably seals a first side surface of each strut to a second side surface of the adjoining strut, thereby separably sealing the struts one to another and closing the intervening apertures 812, which are preferably elongate hexagons in this embodiment. Self-sealing polymer 820 is additionally disposed on an inner surface of each strut, separably sealing the inner surface of the strut to the inner surface of at least one opposing strut, thereby closing narrowed lumen 816.

Struts 811 separate to allow inflation of balloon 830 through central lumen 814 of catheter 810, reseal to maintain inflation, and separate again to allow deflation of the balloon. In this embodiment, the proximal end of catheter 810 is not sealed, and struts 811 may separate in response to inserting a hollow needle into narrowed lumen 816 through the proximal end of catheter 810. When the needle is inserted, struts 811 separate enough to allow passage of the needle but not enough to cause apertures 812 to gap open. The needle may be inserted completely through narrowed lumen 816 and into central lumen 814, thereby allowing passage of a fluid through the needle and into central lumen 814. Alternatively, the needle may be inserted only as far into narrowed lumen 816 as is necessary to cause struts 811 to separate and narrowed lumen 816 to open. In this second example, the fluid passes through the needle, through a portion of narrowed lumen 816, and into central lumen 814.

The polymer sealing the sides of struts 811 together may allow apertures 812 to widen, while still preventing the apertures from gapping when a needle is inserted into narrowed lumen 816. To provide further assurance that apertures 812 will not gap open when a needle is inserted, self-sealing polymer 820 may be further disposed over the outer surface of narrowed region 815. In this example, when struts 811 resume their narrowed configuration, self-sealing polymer 820 fills apertures 812 and narrowed lumen 816 and also forms a layer of polymer over the outer surface of narrowed region 815.

As another measure to prevent gapping or to increase ease of use, an elastic material (not shown) may be coated over the outer surface of narrowed region 815, either directly over the catheter material and any exposed self-sealing polymer or over a layer of self-sealing polymer disposed over the entire outer surface of narrowed region 815. The elastic material may be hydrophilic to ensure that, when the elastic material has been wetted, a second catheter can pass easily there over.

Struts 811 may also separate in response to applying a mechanical force to proximal portion 813 and reseal in response to withdrawing the mechanical force. An adaptor such as that shown in FIG. 6 may be used to apply and withdraw the mechanical force.

The adaptor may be removably mounted on proximal portion 813 of catheter 810. The adaptor has a seal to engage the circumference of catheter 810 distal to proximal portion 813. Engaging the seal establishes a fluid-tight chamber surrounding proximal portion 813. The adaptor is movable between a first position in which a mechanical force is applied to proximal portion 813 and a second position in which the mechanical force is withdrawn. Applying the mechanical force to proximal portion 813 inserts a rod into narrowed lumen 816, separating the struts and opening apertures 812. The adaptor is in fluid communication with a fluid delivery device, and a fluid can now flow into the adaptor and through apertures 812 and central lumen 814 to inflate balloon 830. When the mechanical force is withdrawn, thereby removing the rod from narrowed lumen 816, struts 811 reseal to maintain inflation of the balloon. Once the struts have resealed, the adaptor may be removed and treatment catheters may be serially advanced and withdrawn over the guidewire. To deflate the balloon, the adaptor may be remounted and the mechanical force reapplied to separate struts 811 and allow the fluid to pass out through apertures 812.

Inflatable balloon 830 is operably attached to a distal portion of catheter 810. Inflatable balloon 830 may be made of a suitable material such as thermoplastic polyurethane (TPU) resins, styrene-ethylene-butadiene-styrene (SEBS), PEBAX, or the like.

Although described above in the context of an occlusion guidewire, systems 700 and 800 may be readily adapted to a wide variety of balloon catheters, including those having additional functionalities, structures, or intended uses.

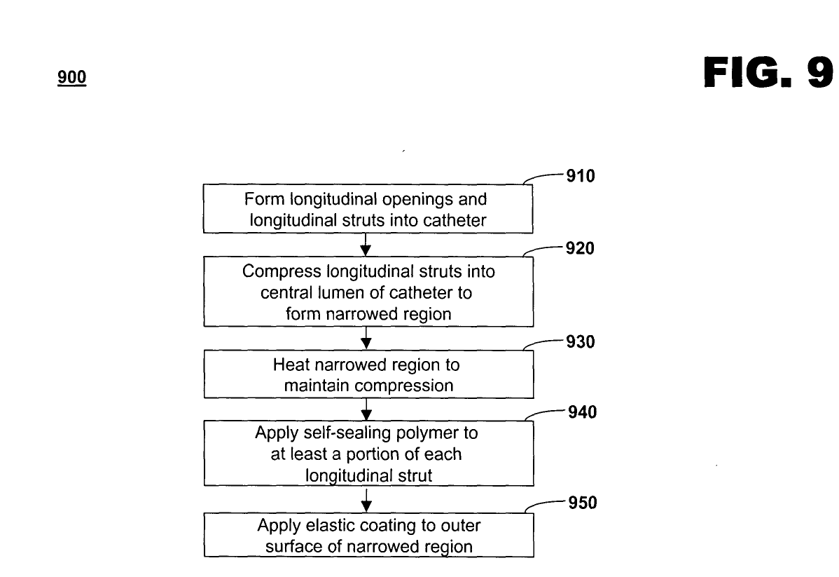

Yet another aspect of the present invention is a method for manufacturing a low-profile catheter valve. FIG. 9 shows a flow diagram of one embodiment in accordance with the present invention at 900.

A plurality of longitudinal apertures and longitudinal struts are formed into a proximal portion of a catheter (Block 910). The longitudinal apertures may be formed by, for example, laser cutting or chemical etching the apertures through the wall of the catheter. The longitudinal struts are formed when the longitudinal apertures are cut or etched into the catheter and are, therefore, interspaced with the longitudinal apertures.

The longitudinal struts may be deformed into a central lumen of the catheter such that a narrowed region is formed in the catheter, the narrowed region having a narrowed lumen (Block 920). This may be accomplished by, for example, placing a removable mandrel within the portion of the catheter containing the longitudinal struts, the mandrel having an outer diameter equal to the desired inner diameter of the narrowed lumen. A radial compressive force may then be applied simultaneously to all of the struts. Radially compressing the longitudinal struts narrows the longitudinal apertures, thus forming a narrowed region in the catheter, the narrowed region having a narrowed lumen. The narrowed region may then be heat-treated, for example in an oven or a heat set block, to maintain or set the shape memory of the radial compression of the longitudinal struts into the central lumen of the catheter (Block 930).

A self-sealing polymer is applied to at least a portion of each longitudinal strut (Block 940). This may be accomplished by, for example, bowing the longitudinal struts into an outwardly extended position and coating the polymer onto at least a portion of each strut. The self-sealing polymer may be polyurethane, silicone, a suitable biocompatible polymer, or the like.

An elastic coating may be applied over an outer surface of the catheter's narrowed region using a suitable method such as spraying or painting (Block 950). The coating material may be applied directly onto the outer surfaces of the longitudinal struts and any self-sealing polymer exposed in the longitudinal apertures, or it may be applied over a layer of self-sealing polymer disposed over the entire outer surface of the narrowed region.

It will be apparent to one skilled in the art that a low-profile catheter valve may be manufactured using just two of the above steps: forming a plurality of longitudinal apertures and longitudinal struts into a proximal portion of a catheter; and applying a self-sealing polymer to at least a portion of each strut, that portion being the side surfaces of the longitudinal struts.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

1. A low-profile catheter valve, comprising:
a catheter having a central lumen, the catheter including a plurality of longitudinal struts and longitudinal apertures, the struts and apertures interspaced around the circumference of a proximal portion of the catheter; and
a self-sealing polymer disposed on at least a portion of each strut, the polymer separably sealing the struts one to another, wherein the struts separate to allow passage of a fluid into or out of the central lumen, and wherein the struts reseal to prevent passage of a fluid into or out of the central lumen.

2. The valve of claim 1 wherein the self-sealing polymer is disposed on at least a first side surface and a second side surface of each strut, and wherein separably sealing the struts one to another comprises separably sealing the first side surface of each strut to the second side surface of an adjoining strut, thereby closing the apertures.

3. The valve of claim 2 wherein a proximal end of the central lumen is sealed proximal to the apertures.

4. The valve of claim 2 wherein the struts are deformed into the central lumen of the catheter such that a narrowed region is formed in the catheter, the narrowed region including a narrowed lumen.

5. The valve of claim 4 wherein the self-sealing polymer is further disposed on at least an inner surface of each strut, and wherein separably sealing the struts one to another further comprises separably sealing the inner surface of each strut to the inner surface of at least one opposing strut, thereby closing the narrowed lumen.

6. The valve of claim 5 further comprising:
an elastic coating disposed over an outer surface of the narrowed region of the catheter.

7. The valve of claim 5 wherein the struts separate in response to inserting a needle into the narrowed lumen, and wherein the struts reseal in response to withdrawing the needle from the narrowed lumen.

8. The valve of claim 5 wherein the struts separate in response to inserting a rod into the narrowed lumen, and wherein the struts reseal in response to withdrawing the rod from the narrowed lumen.

9. The valve of claim 1 wherein the struts separate in response to applying a mechanical force to the proximal portion of the catheter, and wherein the struts reseal in response to withdrawing the mechanical force.

10. The valve of claim 9 further comprising:
an adaptor that may be removably mounted about the proximal portion of the catheter, the adaptor being movable between a first position in which a mechanical force is applied to the proximal portion of the catheter and a second position in which the mechanical force is withdrawn, the adaptor being in fluid communication with a fluid delivery device, the adaptor having a seal to engage the circumference of the catheter distal to the proximal portion of the catheter, wherein engaging the seal establishes a fluid-tight chamber surrounding the proximal portion of the catheter.

11. The valve of claim 9 further comprising:
an adaptor that may be removably mounted on the proximal portion of the catheter, the adaptor being movable between a first position in which a mechanical force is applied to the proximal portion of the catheter and a second position in which the mechanical force is withdrawn, the adaptor being in fluid communication with a fluid delivery device, the adaptor having a first seal to engage the circumference of the catheter distal to the proximal portion of the catheter, the adaptor having a second seal to engage the circumference of the catheter proximal to the proximal portion of the catheter, wherein engaging the first and second seals establishes a fluid-tight chamber surrounding the proximal portion of the catheter.

12. The valve of claim 1 wherein the apertures are narrowly eye-shaped.

13. The valve of claim 1 wherein the apertures are hexagonal.

14. The valve of claim 1 wherein the catheter is a hollow guidewire.

15. The valve of claim 1 wherein an inflatable balloon is operably attached to a distal portion of the catheter, and wherein the struts separate to allow inflation of the balloon through the central lumen of the catheter, reseal to maintain inflation, and separate to allow deflation of the balloon.

16. A system for treating a vascular condition, comprising:
a catheter having a central lumen, the catheter including a plurality of longitudinal struts and longitudinal apertures interspaced around the circumference of a proximal portion of the catheter;
a self-sealing polymer disposed on at least a portion of each strut, the polymer separably sealing the struts one to another; and
an inflatable balloon operably attached to a distal portion of the catheter, wherein the struts separate to allow inflation of the balloon through the central lumen of the catheter, reseal to maintain inflation, and separate to allow deflation of the balloon.

17. The system of claim 16 wherein the catheter is a hollow guidewire.

18. The system of claim 16 wherein the self-sealing polymer is disposed on at least a first side surface and a second side surface of each strut, and wherein separably sealing the struts one to another comprises separably sealing the first side surface of each strut to the second side surface of an adjoining strut, thereby closing the apertures.

19. The system of claim 18 wherein a proximal end of the central lumen is sealed proximal to the apertures.

20. The system of claim 18 wherein the struts are deformed into the central lumen of the catheter such that a narrowed region is formed in the catheter, the narrowed region including a narrowed lumen.

21. The system of claim 20 wherein the self-sealing polymer is further disposed on at least an inner surface of each strut, and wherein separably sealing the struts one to another further comprises separably sealing the inner surface of each strut to the inner surface of at least one opposing strut, thereby closing the narrowed lumen.

22. The system of claim 21 further comprising:
an elastic coating disposed over an outer surface of the narrowed region of the catheter.

23. The system of claim 16 wherein the struts separate in response to applying a mechanical force to the proximal portion of the catheter, and wherein the struts reseal in response to withdrawing the mechanical force.

24. The system of claim 23 further comprising:
an adaptor that may be removably mounted on the proximal portion of the catheter, the adaptor being movable between a first position in which a mechanical force is applied to the proximal portion of the catheter and a second position in which the mechanical force is withdrawn, the adaptor being in fluid communication with a fluid delivery device, the adaptor having a seal to engage the circumference of the catheter distal to the proximal portion of the catheter, wherein engaging the seal establishes a fluid-tight chamber surrounding the proximal portion of the catheter.

25. The system of claim 23 further comprising:
an adaptor that may be removably mounted on the proximal portion of the catheter, the adaptor being movable between a first position in which a mechanical force is applied to the proximal portion of the catheter and a second position in which the mechanical force is withdrawn, the adaptor being in fluid communication with a fluid delivery device, the adaptor having a first seal to engage the circumference of the catheter distal to the proximal portion of the catheter, the adaptor having a second seal to engage the circumference of the catheter proximal to the proximal portion of the catheter, wherein engaging the first and second seals establishes a fluid-tight chamber surrounding the proximal portion of the catheter.

26. The system of claim 21 wherein the struts separate in response to inserting a hollow needle into the narrowed lumen.

27. The system of claim 21 wherein the struts separate in response to inserting a rod into the narrowed lumen.

28. The system of claim 16 wherein the apertures are narrowly eye-shaped.

29. The system of claim 16 wherein the apertures are hexagonal.

30. A method for manufacturing a low-profile catheter valve, comprising:
forming a plurality of longitudinal apertures and longitudinal struts into a proximal portion of a catheter; and
applying a self-sealing polymer to at least a portion of each strut.

31. The method of claim 30 further comprising:
prior to applying the self-sealing polymer, compressing the struts into a central lumen of the catheter such that a narrowed region is formed in the catheter, the narrowed region having a narrowed lumen.

32. The method of claim 31 further comprising:
prior to applying the self-sealing polymer, heating the narrowed region of the catheter to maintain compression of the struts into the central lumen of the catheter.

33. The method of claim 30 wherein applying a self-sealing polymer to at least a portion of each strut comprises:
bowing the struts into an outwardly extending position; and
coating the polymer onto at least a portion of each strut.

34. The method of claim 31 further comprising:
after applying the self-sealing polymer, applying an elastic coating over an outer surface of the narrowed region of the catheter.
